(19) United States
(12) Patent Application Publication
(10) Pub. No.: US 2010/0184026 A1
(43) Pub. Date: Jul. 22, 2010

(54) Companion Diagnostic Assays For Endothelin Receptor Antagonists

(75) Inventors: Richard R. Lesniewski, Pleasant Prairie, WI (US); Dimitri Semizarov, Chicago, IL (US); Charles L. Van Sant, Grayslake, IL (US)

Correspondence Address: VYSIS, INC;PATENT DEPARTMENT, 1300 E TOUHY AVENUE, DES PLAINES, IL, 60018, US

(73) Assignee: ABBOTT LABORATORIES, Abbott Park, IL (US)

(21) Appl. No.: 12/357,588

(22) Filed: Jan. 22, 2009

(51) Int. Cl. C12Q 1/68; G01N 33/00

(52) U.S. Cl. 435/6; 435/7.25

(57) ABSTRACT

Methods for identifying cancer patients eligible to receive endothelin receptor antagonist therapy and for monitoring patient response to endothelin receptor antagonist therapy comprise assessment of the expression levels of at least one of PAI-1, uPA, TGFbeta2, IL-6, IL-8 and OPG in a patient tissue sample. The methods of the invention allow more effective identification of patients to receive endothelin receptor antagonist therapy and of determination of patient response to the therapy.

### FIELD OF THE INVENTION

This invention relates to diagnostic assays useful with endothelin receptor antagonist therapy, and in particular relates to measurement of certain biomarkers that allow identification of patients eligible to receive endothelin receptor antagonist therapy and that permit monitoring of patient response to such therapy.

### BACKGROUND OF THE INVENTION

Endothelin (ET-1) is a 21 amino acid peptide that is produced by endothelial cells. ET is produced by enzymatic cleavage of a Trp-Val bond in the precursor peptide big endothelin (Big ET-1). This cleavage is caused by an endothelin converting enzyme (ECE). Endothelin has been shown to constrict arteries and veins, increase mean arterial blood pressure, decrease cardiac output, increase cardiac contractility in vitro, stimulate mitogenesis in vascular smooth muscle cells in vitro, contract non-vascular smooth muscle including guinea pig trachea, human urinary bladder strips and rat uterus in vitro, increase airway resistance in vivo, induce formation of gastric ulcers, stimulate release of atrial natriuretic factor in vitro and in vivo, increase plasma levels of vasopressin, aldosterone and catecholamines, inhibit release of renin in vitro and stimulate release of gonadotropins in vitro. ET-1 upregulation has also been identified in cancers, including prostate cancer, breast cancer, lung cancer, melanoma and glioma.

Osteoblastic metastases frequently develop in advanced cases of prostate cancer and in several other common malignancies, such as breast cancer, Guise, T. A. and G. R. Mundy, “Cancer and bone”, Endocr. Rev., 1998, 19(1): p. 18-54. The development of metastases at distant sites is driven by interactions between disseminated tumor cells and the host tissue environment. It is believed that the excessive bone growth at the osteoblastic metastatic site is caused by stimulation of the osteoblasts by factors secreted by tumor cells, Id. Several factors have been implicated in this process, including fibroblast growth factors (FGFs) 1 and 2, insulin-like growth factors IGFs) 1 and 2, urokinase-type plasminogen activator (uPA), bone morphogenic proteins (BMPs), and endothelin 1 (ET-1), Nelson, J., et al., “The endothelin axis: emerging role in cancer”, Nat. Rev. Cancer, 2003, 3(2): p. 110-6. ET-1 is secreted by prostate cancer cells and is elevated in plasma from advanced prostate cancer patients, Nelson, J. B., et al., “Identification of endothelin-1 in the pathophysiology of metastatic adenocarcinoma of the prostate”, Nat. Med., 1995. 1(9): p. 944-9. ET-1 has been shown to exert its effects by binding to two cell surface receptors, ETA and ETB, the latter functioning primarily in ligand clearance, Levin, E. R., “Endothelins”, N. Engl. J. Med., 1995, 333(6): p. 356-63.

A significant amount of evidence has been accumulated to support the role of ET-1 in the formation of osteoblastic metastases. Injection of several ET-1-secreting breast cancer cell lines into mice caused formation of osteoblastic metastases, while administration of ABT-627 suppressed the metastatic growth, Yin, J. J., et al., “A causal role for endothelin-1 in the pathogenesis of osteoblastic bone metastases”, Proc. Natl. Acad. Sci. U.S.A., 2003, 100(19): p. 10954-9. However, the precise molecular mechanism whereby ET-1 stimulates osteoblastic bone formation has not been reported.

Antagonistic therapy targeted at the ET receptor has been reported. For example, a selective ETA receptor antagonist, atrasentan, (also called ABT-627), is currently undergoing clinical trials in prostate cancer. The compound extended time to disease progression in patients with metastatic hormone-refractory prostate cancer, Nelson, J., et al., Nat. Rev. Cancer, 2003, 3(2): p. 110-6. ABT-627 is described in U.S. Pat. No. 5,767,144, “Endothelin antagonists”, M. Winn et al., issued Jun. 16, 1998. ET receptor antagonist therapy is important because few options exist to treat metastatic hormone-refractory prostate cancer and the disease is extraordinarily painful.

Because of the potential therapeutic use of ET receptor antagonists, companion diagnostic assays that would identify patients eligible to receive ET receptor antagonist therapy are needed. Additionally, there is a clear need to support this therapy with diagnostic assays using biomarkers that would facilitate monitoring the metastatic load in patients and thus enable monitoring the efficacy of anti-metastatic therapies.

### SUMMARY OF THE INVENTION

The invention provides companion diagnostic assays for use of Endothelin Receptor Antagonist therapy, preferably for cancer treatment. The inventive assays include methods for identifying patients eligible to receive Endothelin Receptor Antagonist therapy and for monitoring patient response to such therapy. These methods comprise assessment in a patient tissue sample of levels of at least one of the biomarkers PAI-1, uPA, TGFbeta2, IL-6, IL-8 and OPG. The inventive methods comprise assessment of the biomarkers in blood, urine or other body fluid samples by immunoassay, proteomic assay or nucleic acid hybridization assays, and in tissue or other cellular body samples by immunohistochemistry or in situ hybridization assays.

In a preferred embodiment, the invention comprises a method for identifying a patient as eligible to receive Endothelin Receptor Antagonist therapy comprising: (a) providing a peripheral blood sample from a patient; (b) determining expression levels in the peripheral blood sample of at least one of PAI-1, uPA, TGFbeta2, IL-6, IL-8 and OPG; (c) classifying the expression level relative to normal peripheral blood level of PAI-1, uPA, TGFbeta2, IL-6, IL-8 and OPG; and (d) identifying the patient as eligible for anti-Endothelin-1 therapy where the patient's blood sample is classified as having elevated levels of at least one of PAI-1, uPA, TGFbeta2, IL-6, IL-8 and OPG.

In another preferred embodiment, the invention comprises a method for identifying a patient as eligible to receive Endothelin Receptor Antagonist therapy comprising: (a) providing a tissue or cellular sample from a patient; (b) contacting the tissue or cellular sample with a labeled antibody or protein capable of binding to at least one of PAI-1, uPA, TGFbeta2, IL-6, IL-8 and OPG; (c) classifying the expression level relative to normal tissue or cellular level of PAI-1, uPA, TGFbeta2, IL-6, IL-8 and OPG; and (d) identifying the patient as eligible for anti-Endothelin-1 therapy where the patient's sample is classified as having elevated levels of at least one of PAI-1, uPA, TGFbeta2, IL-6, IL-8 and OPG.

The invention also comprises a preferred method for monitoring a patient being treated with Endothelin Receptor Antagonist (ETRA) therapy comprising: (a) providing a peripheral blood sample from a patient; (b) measuring expression levels in the peripheral blood sample of at least one of PAI-1, uPA, TGFbeta2, IL-6, IL-8 and OPG; and (c) determining the expression level relative to a patient baseline blood level of PAI-1, uPA, TGFbeta2, IL-6, IL-8 and OPG.

The invention also comprises a reagent kit for an assay for levels of at least one of PAI-1, uPA, TGFbeta2, IL-6, IL-8 and OPG comprising a container comprising at least one labeled antibody or at least one binding protein capable of binding to a biomarker selected from the group consisting of PAI-1, uPA, TGFbeta2, IL-6, IL-8 and OPG.

The invention has significant capability to provide improved selection of patients for ETRA therapy. The assessment of these biomarkers with the invention also allows tracking of individual patient response to the therapy. The inventive assays have utility with any ETRA therapy, including treatment of cancer, coronary angina, cerebral vasospasm, acute and chronic renal failure, gastric ulceration, cyclosporin-induced nephrotoxocity, endotoxin-induced toxicity, asthma, LPL-related lipoprotein disorders, other proliferative diseases, acute or chronic pulmonary hypertension, platelet aggregation, thrombosis, IL-2 mediated cardiotoxicity, colitis, vascular permeability disorders, ischemia-reperfusion injury, Raynaud's disease and migraine.

### BRIEF DESCRIPTION OF THE DRAWINGS

FIG. 1 shows the test data from ELISA assays to the mouse osteoblast cell line MC3T3 for levels of PAI-1, after treatment with endothelin and with endothelin and ABT-627.

FIG. 2 shows the test data from ELISA assays to the mouse osteoblast cell line MC3T3 for levels of OPG, after treatment with endothelin and with endothelin and ABT-627.

FIG. 3 shows the test data from ELISA assays to the mouse osteoblast cell line MC3T3 for levels of IL-6, after treatment with endothelin and with endothelin and ABT-627.

### DETAILED DESCRIPTION OF THE INVENTION

### I. GENERAL

The invention is based on analysis by Applicants of the gene expression signature induced in osteoblasts by endothelin and the impact on the endothelin gene expression signature of an Endothelin Receptor Antagonist. As used herein, an “Endothelin Receptor Antagonist” or “ETRA” refers to a therapeutic compound of any type including small molecule-, antibody-, antisense-, small interfering RNA- or microRNA-based compounds, that binds to the ETA receptor or to ET itself and antagonizes the activity of ET signaling through the ETA receptor. The inventive methods are useful with any known or hereafter developed Endothelin Receptor Antagonist. A preferred ETRA is atrasentan (ABT-627), (2R,3R,4S)-(+)-2-(4-Methoxyphenyl)-4-( 1,3-benzodioxol-5-yl)-1-(N,N-di(n-butyl)aminocarbonylmethyl)-pyrrolidine-3-carboxylic acid.

ETRA therapy has been disclosed for multiple applications, including treatment of cancer, coronary angina, cerebral vasospasm, acute and chronic renal failure, gastric ulceration, cyclosporin-induced nephrotoxocity, endotoxin-induced toxicity, asthma, LPL-related lipoprotein disorders, other proliferative diseases, acute or chronic pulmonary hypertension, platelet aggregation, thrombosis, IL-2 mediated cardiotoxicity, colitis, vascular permeability disorders, ischemia-reperfusion injury, raynaud's disease and migraine. The assays of the invention have potential use with any of these therapies, but are preferred for use with cancer therapy. In particular, the inventive assays are useful with any ETRA therapy for cancers having osteoblastic bone metastasis, including prostate cancer, lung cancer, breast cancer, melanoma and glioma.

The invention comprises diagnostic assays performed on a patient tissue sample of any type or on a derivative thereof, including peripheral blood, tumor or suspected tumor tissues (including fresh frozen and fixed or paraffin embedded tissue), cell isolates such as circulating epithelial cells separated or identified in a blood sample, lymph node tissue, bone marrow and fine needle aspirates. Preferred tissue samples for use herein are peripheral blood, tumor or suspected tumor tissue and bone marrow.

As used herein, PAI-1 (official symbol SERPINE1) means the human plasminogen activator inhibitor 1 gene, which maps to 7q21.3-q22; uPA (official symbol PLAU) means the human urokinase plasminogen activator gene, which maps to 10q24; TGFbeta2 (official symbol TGFB2) means the human transforming growth factor beta 2 gene, which maps to 1q41; IL-6 (official symbol IL6) means the human interleukin 6 gene, which maps to 7p21; IL-8 (official symbol IL8) means the human interleukin 8 gene, which maps to 4q13-q21; and OPG (official symbol TNFRSF11B) means the human osteoprotegerin gene, which maps to 8q24.

Chromosomal loci cited herein are based on Build 35 of the Human Genome Map, as accessed through the University of California Santa Cruz Genome Browser. As used herein, reference to a chromosome locus or band, such as 7q21, refers to all of the loci or sub bands, for example, such as 7q21.1 or7q21.3, within the band.

### II. ETRA BIOMARKERS

The invention comprises assessment in a patient tissue sample of levels of the biomarkers PAI-1, uPA, TGFbeta2, IL-6, IL-8 and OPG, by measurement of these genes at their expressed protein level or by molecular analysis of their chromosomal DNA or translated messenger RNA.

These six gene biomarkers were identified by Applicants through gene expression and ELISA assays as strongly upregulated by endothelin in both mouse and human osteoblasts. Because they are markers of osteoblastic activity and are secreted proteins, they are of particular interest for use in companion diagnostic assays to therapies against metastatic prostate cancer which is exemplified by extensive osteoblastic activity. Of these six, a preferred biomarker is OPG because of its more direct tie to osteoblastic activity; OPG is known to suppress osteoclastogenesis by interfering with RANK/RANKL interactions, Hofbauer, L. C. and A. E. Heufelder, “Clinical review 114: hot topic. The role of receptor activator of nuclear factor-kappaB ligand and osteoprotegerin in the pathogenesis and treatment of metabolic bone diseases”, J. Clin. Endocrinol. Metab., 2000, 85(7): p. 2355-63, and elevated OPG concentrations have been detected in bone metastases of prostate cancer relative to the primary tumors and nonosseous metastases, Brown, J. M., et al., “Osteoprotegerin and RANK ligand expression in prostate cancer”, Urology, 2001, 57(4): p. 611-6. OPG in plasma levels may directly relate to the increased bone growth due to metastases.

Applicants have assessed the expression in the mouse MC3T3 osteoblast cell line of PAI-1, OPG and IL-6, using commercially available ELISA assay kits. The data showed that treatment of this cell line with endothelin strongly induced expression of each of PAI-1, OPG and IL-6, and that treatment with endothelin in the presence of the ETRA ABT-627 resulted in substantial suppression of this expression. Hence, measurement of these biomarkers is indicative of endothelin expression and suitability for treatment with an ETRA. Applicants attempted to determine PAI-1 and OPG levels in 43 plasma samples from patients participating in a clinical trial of ABT-627. However, the number of samples available was insufficient to provide statistical significance for the data. No adverse or positive trends in the data were seen concerning the use of PAI-1 or OPG as markers of ABT-627 response.

### III. ASSAYS

The inventive assays include assays both to select patients eligible to receive ETRA therapy and assays to monitor patient response. These assays can be performed by protein assay methods and by nucleic acid assay methods. Any type of either protein or nucleic acid assays can be used. Protein assay methods useful in the invention are well known in the art and comprise (i) immunoassay methods involving binding of a labeled antibody or protein to the expressed protein or fragment of PAI-1, uPA, TGFbeta2, IL-6, IL-8 or OPG, (ii) mass spectrometry methods to determine expressed protein or fragments of these biomarkers, and (iii) proteomic based or “protein chip” assays. Useful immunoassay methods include both solution phase assays conducted using any format known in the art, such as, but not limited to, an ELISA format, a sandwich format, a competitive inhibition format (including both forward or reverse competitive inhibition assays) or a fluorescence polarization format, and solid phase assays such as immunohistochemistry (referred to as “IHC”).

IHC methods are particularly preferred assays. IHC is a method of detecting the presence of specific proteins in cells or tissues and consists of the following steps: 1) a slide is prepared with the tissue to be interrogated; 2) a primary antibody is applied to the slide and binds to specific antigen; 2) the resulting antibody-antigen complex is bound by a secondary, enzyme-conjugated, antibody; 3) in the presence of substrate and chromogen, the enzyme forms a colored deposit (a “stain”) at the sites of antibody-antigen binding; and 4) the slide is examined under a microscope to identify the presence of and extent of the stain.

Nucleic acid assay methods useful in the invention are also well known in the art and comprise (i) in situ hybridization assays to intact tissue or cellular samples to detect mRNA levels or chromosomal DNA changes, (ii) microarray hybridization assays to detect mRNA levels or chromosomal DNA changes, (iii) RT-PCR assays or other amplification assays to detect mRNA levels or (iv) PCR or other amplification assays to detect chromosomal DNA changes. Assays using synthetic analogs of nucleic acids, such as peptide nucleic acids, in any of these formats can also be used.

The assays of the invention are used to identify elevated levels of at least one of the biomarkers PAI-1, uPA, TGFbeta2, IL-6, IL-8 and OPG for both response prediction and for monitoring patient response to ETRA therapy. Assays for response prediction are run before therapy selection and patients with elevated leves are eligible to receive ETRA therapy. For monitoring patient response, the assay is run at the initiation of therapy to establish baseline levels of the biomarker in the tissue sample. The same tissue is then sampled and assayed and the levels of the biomarker compared to the baseline. Where the levels remain the same or decrease, the therapy is likely being effective and can be continued. Where significant increase over baseline level occurs, the patient may not be responding. For example, the percent of total cell or number of cells in the sample showing expression of the biomarker as measured by IHC or showing copy number gain as measured by in situ hybridization can be measured at baseline and then periodically during therapy.

The invention also comprises assays for identifying a patient with cancer as eligible to receive anti-Endothelin-1 therapy comprising: (a) providing a tumor sample from a cancer patient; (b) determining expression levels in the tumor sample of at least 10 different genes by nucleic acid analysis; (c) classifying the expression level relative to normal tissue level of the at least 10 different genes; and (d) identifying the cancer patient as eligible for anti-Endothelin-1 therapy where the cancer patient's tumor sample is classified as having elevated levels of at least one of the 10 different genes. In this embodiment, it is preferred that the at least 10 different genes include each of PAI-1, uPA, TGFbeta2, IL-6, IL-8 and OPG.

### IV. IMMUNOASSAYS

Immunoassays are preferred and IHC methods are particularly preferred. IHC is a method of detecting the presence of specific proteins in cells or tissues and consists of the following steps: 1) a slide is prepared containing the tissue to be interrogated; 2) a primary antibody is applied to the slide and binds to specific antigen; 3) the resulting antibody-antigen complex is bound by a detection antibody which is labeled (for example with a conjugated enzyme; 4) the binding of the antibody to its target antigen is detected by examining the slide, generally under a microscope to identify the presence of and extent of the binding.

Any suitable antibodies or binding proteins that bind to the particular biomarker can be used. Monoclonal antibodies are preferred, and suitable antibodies or assay kits are available as follows: anti-human PAI-1 assay kit from American Diagnostica (New York, N.Y.), anti-human OPG assay kit from R& D Systems (Minneapolis, Minn.), anti-human monoclonal antibody to IL-6 and to IL-8 from R&D Systems and Abcam, Inc. (Cambridge, Mass.), anti-human polyclonal antibody, unconjugated, to TGF beta 2 from R&D Systems and Endogen (Rockford, Ill.), and antibody to uPA from American diagnostica (Stamford, Conn.) and anti-human monoclonal antibody, unconjugated, to uPA from GeneTex (San Antonio, Tex.). The biomarker-antibody/protein immune complexes formed in these assays can be detected using any suitable technique. Any suitable label can be used. The selection of a particular label is not critical, but the chosen label must be capable of producing a detectable signal either by itself or in conjunction with one or more additional substances.

Useful detectable labels, their attachment to antibodies and detection techniques therefore are known in the art. Any detectable label known in the art can be used. For example, the detectable label can be a radioactive label, such as, ³H, ¹²⁵I, ³⁵S, ¹⁴C, ³²P, ³³P, an enzymatic label, such as horseradish peroxidase, alkaline peroxidase, glucose 6-phosphate dehydrogenase, etc., a chemiluminescent label, such as, acridinium derivatives, luminol, isoluminol, thioesters, sulfonamides, phenanthridinium esters, etc. a fluorescence label, such as, fluorescein (5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemi (1996), which is a combined handbook and catalogue published by Molecular Probes, Inc., Eugene, Oreg., each of which is incorporated herein by reference. Preferred labels for use with the invention are chemiluminscent labels such as acridinium-9-carboxamide. Additional detail can be found in Mattingly, P. G., and Adamczyk, M. (2002) Chemiluminescent N-sulfonylacridinium-9-carboxamides and their application in clinical assays, in Luminescence Biotechnology: Instruments and Applications (Dyke, K. V., Ed.) pp 77-105, CRC Press, Boca Raton.

The detectable label can be bound to the analyte or antibody either directly or through a coupling agent. An example of a coupling agent that can be used is EDAC (1-ethyl-3-(3-dimethylaminopropyl)carbodiimide, hydrochloride) that is commercially available from Sigma-Aldrich (St. Louis, Mo.). Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as, N10-(3-sulfopropyl)-N-(3-carboxypropyl)-acridinium-9-carboxamide, otherwise known as CPSP-Acridinium Ester or N10-(3-sulfopropyl)-N-(3-sulfopropyl)-acridinium-9-carboxamide, otherwise known as SPSP-Acridinium Ester.

After formation of the labeled complex, the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. For solution phase immunoassays, once the amount of the label in the complex has been quantified, the concentration of biomarker in the test sample is determined by use of a standard curve that has been generated using serial dilutions of the biomarker of known concentration. Other than using serial dilutions of the biomarker, the standard curve can be generated gravimetrically, by mass spectroscopy and by other techniques known in the art.

For the preferred IHC assays, detection of the antibody-antigen binding is preferably done using a conjugated enzyme label attached to a secondary binding antibody, such as horseradish perioxidase. These enzymes in the presence of colored substrate, produce at the site of the binding a colored deposit, called the stain, which can be identified under a light microscope. The site and extent of the staining is then identified and classified. In addition to manual inspection of the slide, automated IHC imaging techniques are known to the art and can be used.

### V. NUCLEIC ACID TYPE ASSAYS

The invention comprises detection of the biomarker levels by hybridization assays using detectably labeled nucleic acid-based probes, such as deoxyribonucleic acid (DNA) probes or protein nucleic acid (PNA) probes, or unlabeled primers which are designed/selected to hybridize to the specific designed chromosomal target. The unlabeled primers are used in amplification assays, such as by polymerase chain reaction (PCR), in which polymerases amplify the target nucleic acid sequence for subsequent detection. The detection probes used in PCR or other amplification assays are preferably fluorescent, and still more preferably, detection probes useful in “real-time PCR”. Fluorescent labels are also preferred for use in situ hybridization but other detectable labels commonly used in hybridization techniques, e.g., enzymatic, chromogenic and isotopic labels, can also be used. Useful probe labeling techniques are described in Molecular Cytogenetics: Protocols and Applications, Y.-S. Fan, Ed., Chap. 2, “Labeling Fluorescence In Situ Hybridization Probes for Genomic Targets”, L. Morrison et.al., p. 21-40, Humana Press, ©2002, incorporated herein by reference.

Reverse transcription PCR (RT-PCR) assays are a well-known amplification method to detect level of mRNA's in a sample, and are useful in the invention. In this aspect, any suitable reverse transcriptase method is used to produce a mRNA population from the patient sample. The mRNA population is then amplified by PCR using a pair of primers specific to at least one of PAI-1, uPA, TGFbeta2, IL-6, IL-8 or OPG, or by multiplex PCR, using multiple pairs of primers. Any primer sequence for the biomarkers can be used.

Suitable probes for use in the in situ hybridization methods utilized with the invention fall into two broad groups: chromosome enumeration probes, i.e., probes that hybridize to a chromosomal region, usually a repeat sequence region, and indicate the presence or absence of an entire chromosome, and locus specific probes, i.e., probes that hybridize to a specific locus on a chromosome and detect the presence or absence of a specific locus As is well known in the art, a chromosome enumeration probe can hybridize to a large chromosome-specific tandemly repeated sequence, which is usually located at or near the centromeres. For example, a chromosome enumeration probe can hybridize with alpha repeat or tandem repeat sequences. Centromere fluorescence in situ hybridization probes are commercially available from Abbott Molecular (Des Plaines, Ill.).

The preferred hybridization probes employ directly labeled fluorescent probes, such as described in U.S. Pat. No. 5,491,224. Useful locus specific probes can be produced in any manner, but preferably will hybridize to a target stretch of chromosomal DNA at the target locus of at least 100,000 bases long, and to use unlabeled blocking nucleic acid, as disclosed in U.S. Pat. No. 5,756,696, herein incorporated by reference, to avoid non-specific binding of the probe. Clones suitable for use to manufacture FISH probes can be identified using the Human Genome Map, as accessed through the University of California Santa Cruz Genome Browser, to identify clone coordinates, and then screening clone libraries for clones mapping to the selected coordinates. It is also possible to use unlabeled, synthesized oligomeric nucleic acid or peptide nucleic acid as the blocking nucleic acid or as the centromeric probe. For targeting the particular gene locus, it is preferred that the probes span the entire genomic coding locus of the gene. Examples of fluorophores that can be used in the in situ hybridization methods described herein are: 7-amino-4-methylcoumarin-3-acetic acid (AMCA), Texas Red™ (Molecular Probes, Inc., Eugene, Oreg.); 5-(and-6)-carboxy-X-rhodamine, lissamine rhodamine B, 5-(and-6)-carboxyfluorescein; fluorescein-5-isothiocyanate (FITC); 7-diethylaminocoumarin-3-carboxylic acid, tetramethyl-rhodamine-5-(and-6)-isothiocyanate; 5-(and-6)-carboxytetramethylrhodamine; 7-hydroxy-coumarin-3-carboxylic acid; 6-[fluorescein 5-(and-6)-carboxamido]hexanoic acid; N-(4,4-difluoro-5,7-dimethyl-4-bora-3a,4a diaza-3-indacenepropionic acid; eosin-5-isothiocyanate; erythrosine-5-isothiocyanate; 5-(and-6)-carboxyrhodamine 6G; and Cascade™ blue aectylazide (Molecular Probes, Inc., Eugene, Oreg.).

The use of a pair of probes allows the determination on a cell-by-cell basis of whether gene amplification, ie. a ratio of the number of the gene locus probe signals to the centromere probe signals in each cell that is greater than 2, exists, or whether gain of the entire chromosome has occurred, ie. a ratio of the number of the gene locus probe signals to the centromere probe signals in each cell of 1/1 to less than 2/1, but with more than the normal number of two gene locus probe signals. Samples that are classified as amplified or having three or more gene locus probe signals are identified as eligible for ETRA therapy.

### VI. SAMPLE PROCESSING

The preferred tissue samples for use herein are peripheral blood, tumor or suspected tumor tissue and bone marrow, and can be processed by conventional methods for IHC, other immunoassays, in situ hybridization or other nucleic acid assays. The assays can also be performed on cell nuclei isolated from a tissue sample. For the preferred IHC assays, a paraffin embedded tumor tissue sample or bone marrow sample is fixed on a glass microscope slide and deparaffinized with a solvent, typically xylene. A conventional antigen retrieval step is then used followed by application of the labeled antibody. Conventional IHC protocols useful in the invention can be found on the Internet web site of IHC World at ihcworld.com.

### VII. INSTRUMENTATION

Any suitable instrumentation or automation can be used in the performance of the inventive assays. The preferred IHC assays can be done on the automated staining systems commercially available from Ventana Medical Systems, BioGenex, DakoCytomation or Vision Biosystems. Solution phase immunoassays can be done in an automated fashion, such as on the Architect® (a registered trademark of Abbott Laboratories, Abbott Park, Ill.) system, which uses chemiluminescense detection of sandwich hybridization and competitive immunoassays. The assays can also be carried out in a miniaturized format, such as in a Lab-on-a-Chip device and system. PCR based assays can be performed on the m2000 instrument system (Abbott Molecular, Des Plaines, Ill.). Automated imaging can be employed for both the preferred IHC assays and for in situ hybridization assays.

### VIII. ASSAY KITS

In another aspect, the invention comprises immunoassay kits for the detection of which kits comprise a labeled antibody or labeled protein specific for binding to at least one of the biomarkers PAI-1, uPA, TGFbeta2, IL-6, IL-8 and OPG. These kits may also include an antibody capture reagent or antibody indicator reagent useful to carry out a sandwich immunoassay. Preferred kits of the invention comprise containers containing, respectively, at least one antibody capable of binding specifically to at least one of the biomarkers PAI-1, uPA, TGFbeta2, IL-6, IL-8 and OPG, and a control composition comprising at least one of the biomarkers PAI-1, uPA, TGFbeta2, IL-6, IL-8 and OPG. Any suitable control composition for the particular biomarker assay can be included in the kits of the invention. The control compositions generally comprise the biomarker to be assayed for along with any desirable additives.

### VII. EXAMPLES

### Example 1

A genome-wide view of ET signaling was assessed using gene expression microarrays.

Cell Culture and Reagents.

Mouse MC3T3 pre-osteoblastic cells (subclone 4) were purchased from ATCC (Manassis, Va.) and propagated in αMEM media without ascorbic acid (Invitrogen, Carlsbad, Calif.) supplemented with 10% FBS (Invitrogen). Human Mesenchymal Stem Cells (MSCs) were purchased from Cambrex (Walkersville, Md.) and propagated according in MSCGM™ media (Cambrex). To initiate differentiation of the MSC into human osteoblasts, the growth media was replaced by osteogenic differentiation medium (OGM, Cambrex).

Cell Growth and Treatment.

Mouse preosteoblastic MC3T3 cells as well as primary human osteoblasts were treated with ET, from Sigma (St. Louis, Mo.), for 2, 4, and 6 hours in the absence or presence of the ETₐ receptor antagonist ABT-627, from Abbott Laboratories (Abbott Park, Ill.). The drug was added 1 hour prior to the addition of ET.

Microarray Analysis of Gene Expression.

Total RNA was extracted from the treated cell lines and purified on RNeasy columns (Qiagen, Valencia, Calif.). Labeled cRNA was prepared according to the microarray manufacturer Affymetrix's protocol and hybridized to either mouse 430A 2.0 or human U133A 2.0 arrays (Affymetrix, Santa Clara, Calif.). Gene expression fold changes for each treatment were calculated by combining three biological replicates for each treatment using the Rosetta Resolver's Affymetrix error model software and building a ratio from the resulting values. All genes regulated ≧1.5-fold with a p-value of ≦0.01 were retained for further analysis. Conventional two-dimensional clustering was then performed by using the agglomerative hierarchical clustering algorithm. The Euclidean distance was used as the similarity metric.

From the two-dimensional hierarchical clustering of the gene expression signatures for ET, a significant number of genes were induced at all three timepoints, while pre-treatment with the ABT-627 abrogated almost all of these gene induction events. The antagonist alone caused very few gene expression changes. The ET-1 treatments clustered together because of the similarity of the signatures, while the rest of the treatments show a random clustering pattern because of the insignificant number of genes regulated. Table 1 summarizes the microarray data for the mouse osteoblasts. ET-1 induced 608 genes at 2 hours; the number of upregulated genes decreased with time. The overwhelming majority of the gene induction events was abrogated by ABT-627, indicating that ET-1 signals exclusively through the ETA receptor. The microarray experiment in primary human osteoblasts revealed very similar statistics.

TABLE 1

| | Time point | | |
|---|---|---|---|
| | 2 hr | 4 hr | 6 hr |
| Induced by ET | 608 | 472 | 194 |
| # Genes Blocked by | 581 | 390 | 189 |
| ABT-627 | (96%) | (83%) | (97%) |
| # Genes Down- | 423 | 295 | 95 |
| regulated by ET | | | |
| # Genes Blocked by | 403 | 262 | 93 |
| ABT-627 | | | |

Pathway analysis of the ET-1 signature in osteoblastic cells revealed several dominant motifs. Firstly, an osteoblastic maturation motif was represented in the ET-1 expression signature by such genes as osteoprotegerin (OPG), COX-2, Dmp1, Tgfbi, CTGF, and Kruppel-like factor 10 (Klf10). Because of the early timepoints chosen, the genes induced are implicated primarily into the differentiation process, rather than the maintenance of the mature osteoblastic phenotype. The induction of these genes by ET-1 was blocked by pre-treatment with ABT-627. Secondly, an invasion signature included expression of uroplasminogen activator (uPA), uroplasminogen activator receptor (uPAR), plasminogen activator inhibitor (PAI-1), TGFbeta2, IL-6, IL-8, and CTGF. The products of these genes have been previously implicated in metastasis and shown to be elevated in metastatic cancer patients, see George, D. J., et al., “The prognostic significance of plasma interleukin-6 levels in patients with metastatic hormone-refractory prostate cancer: results from cancer and leukemia group B 9480”, Clin. Cancer Res., 2005, 11(5): p. 1815-20; Benoy, I. H., et al., “Increased serum interleukin-8 in patients with early and metastatic breast cancer correlates with early dissemination and survival”, Clin. Cancer Res., 2004, 10(21): p. 7157-62; and Kang, Y., et al., “A multigenic program mediating breast cancer metastasis to bone”, Cancer Cell, 2003, 3(6): p. 537-49. Finally, the third theme in the ET signature was suppression of apoptosis. This group comprised Nur77, Flt1, and NFATc1. Again, these genes were induced by ET, and the induction was blocked by ABT-627.

### Example 2

Several of the genes identified in Example 1 as strongly upregulated by ET-1 in both mouse and human osteoblasts code for secreted proteins. Specifically, two members of the plasminogen system (PAI-1 and uPA), TGFbeta2, and two interleukins (IL-6, and IL-8) were induced. In this Example 2, ELISA-based assays were used to demonstrate secretion by osteoblasts of PAI-1, OPG and IL-6.

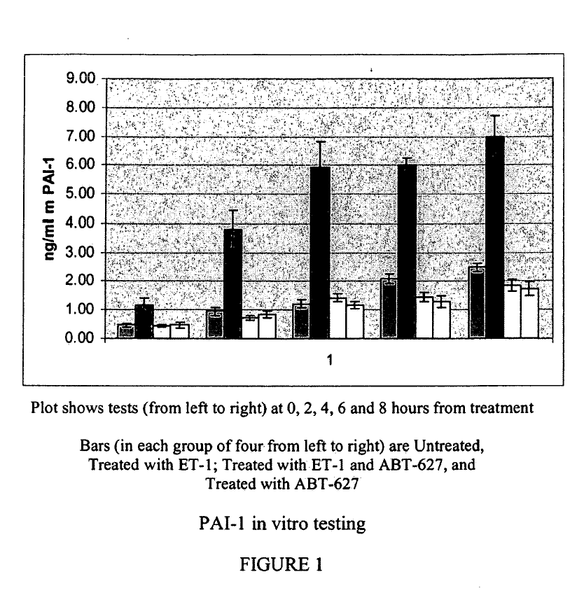

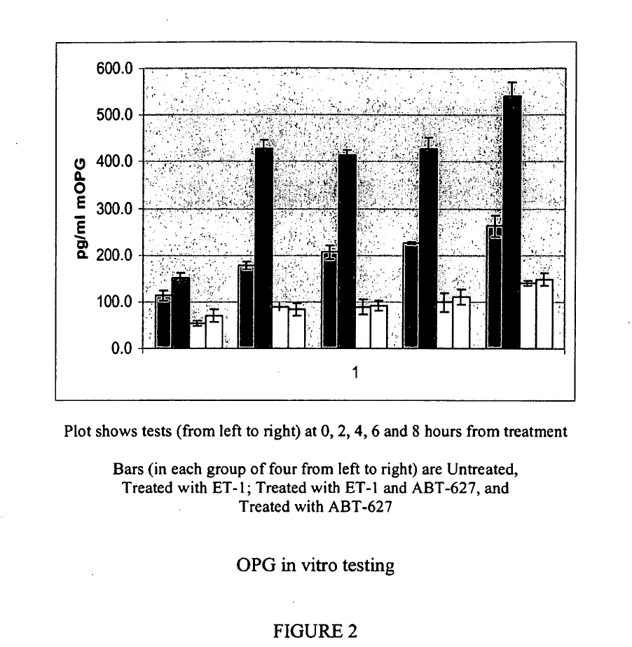

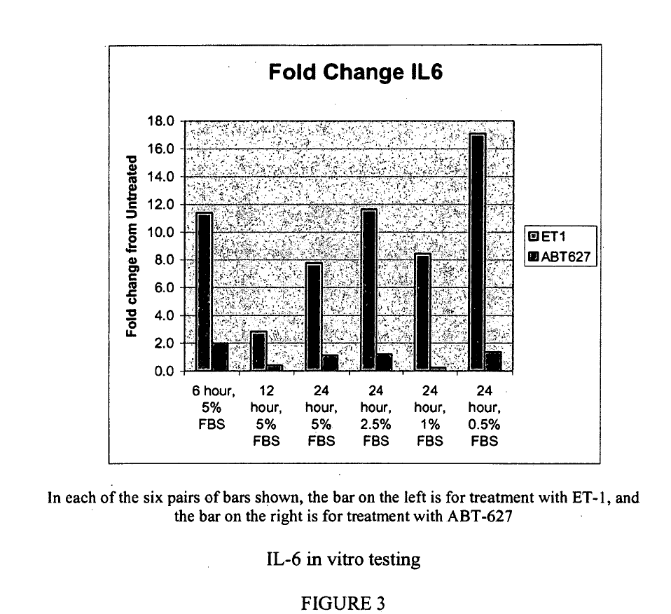

Mouse MC3T3 osteoblast cells were propagated as set out above, and at times indicated were harvested and spun at 250×g for 10 minutes at room temperature. The clarified supernatants were aliquoted and frozen until analyzed. 200 microliters of each sample was tested in quadruplicate by commercially available ELISA assay kits for PAI-1 (Molecular Innovations, Southfield, Mich.), OPG (Biomedica, San Diego, Calif.) and IL-6 (Ray Biotech, Norcross, Ga.). The ELISA tests were performed according to the manufacturer's instructions. Data from these tests are shown in FIG. 1 (PAI-1), FIG. 2 (OPG) and FIG. 3 (IL-6).

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

What is claimed is:

1. A method for identifying a patient with cancer as eligible to receive anti-Endothelin-1 therapy comprising: (a) providing tissue sample from a patient; (b) determining level in the tissue sample of at least one of PAI-1, uPA, TGFbeta2, IL-6, IL-8 and OPG; (c) classifying the level relatives to levels in normal tissue of PAI-1, uPA, TGFbeta2, IL-6, IL-8 and OPG; and (d) identifying the patient as eligible for anti-Endothelin-1 therapy where the patient's sample is classified as having an elevated level of at least one of PAI-1, uPA, TGFbeta2, IL-6, IL-8 and OPG.

2. The method of claim 1 wherein the tissue sample is a peripheral blood sample from a patient with a cancer selected from the group consisting of prostate carcinoma, breast carcinoma, lung carcinoma, melanoma and glioma.

3. The method of claim 1 wherein the tissue sample is a peripheral blood sample and expression level in the peripheral blood sample of at least one of PAI-1, uPA, TGFbeta2, IL-6, IL-8 and OPG is determined by immunoassay.

4. The method of claim 1 wherein the tissue sample is a peripheral blood sample and the expression level of at least one of PAI-1, uPA, TGFbeta2, IL-6, IL-8 and OPG is determined by proteomic analysis.

5. The method of claim 1 the tissue sample is a peripheral blood sample and wherein the expression level of at least one of PAI-1, uPA, TGFbeta2, IL-6, IL-8 and OPG is determined by mRNA analysis.

6. The method of claim 1 the tissue sample is a peripheral blood sample and wherein the expression level of OPG is determined in a patient with prostate cancer by immunoassay.

7. A method for monitoring a patient being treated with anti-Endothelin-1 therapy comprising: (a) providing a peripheral blood sample from a cancer patient; (b) measuring expression levels in the peripheral blood sample of at least one of PAI-1, uPA, TGFbeta2, IL-6, IL-8 and OPG; and (c) determining the expression level relative to a patient baseline blood level of PAI-1, uPA, TGFbeta2, IL-6, IL-8 and OPG.

8. The method of claim 7 wherein the cancer is selected from the group consisting of prostate carcinoma, melanoma, glioma, and lung carcinoma.

9. The method of claim 7 wherein the expression levels of at least one of PAI-1, uPA, TGFbeta2, IL-6, IL-8 and OPG is measured by immunoassay.

10. The method of claim 7 wherein the expression levels of at least one of PAI-1, uPA, TGFbeta2, IL-6, IL-8 and OPG is measured by proteomic analysis.

11. The method of claim 7 wherein the expression levels of at least one of PAI-1, uPA, TGFbeta2, IL-6, IL-8 and OPG is measured by mRNA analysis.

12. The method of claim 7 wherein the expression level of OPG is measured in a patient with prostate cancer by immunoassay.

13. A method for identifying a patient with cancer as eligible to receive anti-Endothelin-1 therapy comprising: (a) providing a tumor sample from a cancer patient; (b) determining expression levels of at least one of PAI-1, uPA, TGFbeta2, IL-6, IL-8 and OPG by immunohistochemistry; (c) classifying the expression level relative to normal tissue level of PAI-1, uPA, TGFbeta2, IL-6, IL-8 and OPG; and (d) identifying the cancer patient as eligible for anti-Endothelin-1 therapy where the cancer patient's tumor sample is classified as having elevated levels of at least one of PAI-1, uPA, TGFbeta2, IL-6, IL-8 and OPG.

14. The method of claim 13 wherein the cancer is selected from the group consisting of prostate carcinoma, melanoma, glioma, and lung carcinoma.

15. The method of claim 13 wherein the expression level of OPG is determined in a patient with prostate cancer.

16. A method for identifying a patient with cancer as eligible to receive anti-Endothelin-1 therapy comprising: (a) providing a tumor sample from a cancer patient; (b) determining expression levels of at least 10 different genes by nucleic acid analysis; (c) classifying the expression level relative to normal tissue level of the at least 10 different genes; and (d) identifying the cancer patient as eligible for anti-Endothelin-1 therapy where the cancer patient's tumor sample is classified as having elevated levels of at least one of the 10 different genes.

17. The method of claim 16 wherein the at least 10 different genes include each of PAI-1, uPA, TGFbeta2, IL-6, IL-8 and OPG.

18. The method of claim 16 wherein the cancer is selected from the group consisting of prostate carcinoma, melanoma, glioma, and lung carcinoma.

19. The method of claim 16 wherein the expression level is determined in a patient with prostate cancer.
